(19) United States
(12) Patent Application Publication
(10) Pub. No.: US 2005/0002872 A1
(43) Pub. Date: Jan. 6, 2005

(54) No sweat

(76) Inventor: Samuel Elliott Katz, Atlanta, GA (US)

Correspondence Address: Sam Katz, Suite 106, 333 Sandy Springs Circle, Atlanta, GA, 30328, US

(21) Appl. No.: 10/609,718

(22) Filed: Jul. 1, 2003

(51) Int. Cl. A61K 35/78; A61K 7/00; A61K 7/32

(52) U.S. Cl. 424/47; 424/65; 424/773

(57) ABSTRACT

Aerosol antiperspirant deodorant spray comprising of: A.) corn starch, arrowroot powder, baking soda and/or at least a propellant and B.) a carrying agent Formula Combination: Currently, the combination of Corn Starch, Arrowroot, and Baking Soda are milled down to a micron level around 5, where the powders are then blended together and placed in a can with a combination of a propellant and a carrying agent such as alcohol. Combine the above ingredients together in a 100 Gram can with a 3.5 oz weight and having a 6.02 weight in liquid volume.

The product is comprised of the following specifications:
1.) 100 Gram Can
2.) 3.5 Oz weight
3.) 6.02 Weight in Liquid Volume
4.) 69.25% Propellant
5.) 14.25% of SDA-40 190
6.) 5% Corn Starch
7.) 5% Arrowroot Powder
8.) 5% Baking Soda
9.).5% of Propylene Glycol

### BREIF SUMMARY OF THE INVENTION:

“No Sweat” is an antiperspirant type powder, similar to Baby Powder, however concentrated in a spray. Baby Powder and Talcum powder end up all over the floor and on clothes. No Sweat was designed with eliminating those two common problems associated with the above mentioned products. No Sweat allows one to spray the product on areas associated with the use of Baby Powder and talcum powder. After the product is shaken well, and sprayed from a safe distance of at least a 12 inches, the alcohol will evaporate in 5 seconds from the skin leaving the powder in the exact area the spray was directed; leaving no mess.

1. I claim that this is an Aerosol antiperspirant deodorant spray comprising of:
C.) corn starch, arrowroot powder, baking soda and/or at least a propellant and
D.) a carrying agent
Formula Combination:
Currently, the combination of Corn Starch, Arrowroot, and Baking Soda are milled down to a micron level around 5, where the powders are then blended together and placed in a can with a combination of a propellant and a carrying agent such as alcohol. Combine the above ingredients together in a 100 Gram can with a 3.5 oz weight and having a 6.02 weight in liquid volume.
